(19) United States
(12) Patent Application Publication
(10) Pub. No.: US 2025/0135206 A1
(43) Pub. Date: May 1, 2025

(54) SYSTEMS AND METHODS FOR UTILIZING POWER SPECTRA FEATURES IN ADAPTIVE NEUROMODULATION APPLICATIONS

(71) Applicant: Boston Scientific Neuromodulation Corporation, Valencia, CA (US)

(72) Inventors: Andrew James Haddock, Los Angeles, CA (US); G. Karl Steinke, Valencia, CA (US); Raul Enrique Serrano Carmona, Los Angeles, CA (US)

(21) Appl. No.: 18/905,413

(22) Filed: Oct. 3, 2024

Related U.S. Application Data
(60) Provisional application No. 63/546,067, filed on Oct. 27, 2023.

(51) Int. Cl. A61N 1/36; A61N 1/05; A61N 1/372

(52) U.S. Cl. CPC A61N 1/36139; A61N 1/0534; A61N 1/36067; A61N 1/37247

(57) ABSTRACT

This document discusses a neurostimulation device to monitor electrical neural activity when connected to implantable electrodes. The neurostimulation device includes a sensing circuit configured to sense sensing a local field potential (LFP) signal of a patient when connected to implantable electrodes and signal processing circuitry operatively coupled to the sensing circuit. The signal processing circuitry is configured to compute a power spectral density (PSD) of the sensed LFP signal, compute a slope of the PSD of the sensed LFP signal, and determine a physiological state of the patient using the computed slope of the PSD of the sensed LFP signal.

### CLAIM OF PRIORITY

This application claims the benefit of U.S. Provisional Application No. 63/546,067, filed on Oct. 27, 2023, which is hereby incorporated by reference in its entirety.

### TECHNICAL FIELD

This document relates generally to medical devices and more particularly to a system for neurostimulation.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device can be used to program the implantable neurostimulator with stimulation parameters controlling the delivery of the neurostimulation energy.

In one example, the neurostimulation energy is delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation systems may offer many programmable options for the parameters of the neurostimulation to customize the neurostimulation therapy for a specific patient. For some types of neurostimulation (e.g., DBS) the efficacy of the neurostimulation for the patient may depend on the neurostimulation system performing adaptive or decision-making algorithms to optimize the neurostimulation therapy. The decision-making algorithms performed by the neurostimulation system may use biomarkers as indicators to make therapy decisions or other decisions related to treatment. The decision-making performance of the neurostimulation system can be compounded by biomarker indications that are not relevant therapeutically.

### SUMMARY

Neurostimulation, also referred to as neuromodulation, can involve delivering electrical neurostimulation energy in the form of electrical neurostimulation pulses to treat a neurological condition of the patient. Biomarkers of neural activity can be used as feedback for adaptive neuromodulation approaches. However, reliance on a single measure of neural activity may lead to a poorly performing adaptive algorithm due to non-therapeutically relevant events affecting the measurements.

Example 1 includes subject matter (such as a method of operating a medical device) comprising sensing a local field potential (LFP) signal of a patient using the medical device; determining, by the medical device, a power spectral density (PSD) of the sensed LFP signal and a slope of the PSD of the sensed LFP signal; and determining a physiological state of the patient using the slope of the PSD of the sensed LFP signal.

In Example 2, the subject matter of Example 1 optionally includes delivering electrical neurostimulation therapy to the patient using the medical device; detecting a movement state of the patient using the slope of the PSD of the sensed LFP signal, wherein the movement state is indicative of tremor movement of the patient; and changing a neurostimulation therapy parameter based on the detected movement state of the patient.

In Example 3, the subject matter of one or both of Examples 1 and 2 optionally includes determining a medication state of the patient using the slope of the PSD of the sensed LFP signal; and generating a prompt related to medication of the patient based on the determined medication state of the patient.

In Example 4, the subject matter of one or any combination of Examples 1-3 optionally includes delivering electrical neurostimulation therapy to the patient using the medical device; detecting a sleep state of the patient using the slope of the PSD of the sensed LFP signal, wherein the sleep state is indicative of depth of the sleep of the patient; and changing a neurostimulation therapy parameter based on the detected sleep state of the patient.

In Example 5, the subject matter of one or any combination of Examples 1-4 optionally includes delivering electrical neurostimulation therapy to the patient using the medical device; detecting a change in impairment state of the patient using the slope of the PSD of the sensed LFP signal; and changing a neurostimulation therapy parameter based on the detected change in impairment state of the patient.

In Example 6, the subject matter of one or any combination of Examples 1-5 optionally includes sensing a spinal LFP signal from spinal nerve tissue of the patient; determining the slope of the PSD of the sensed spinal LFP signal; and determining a change in a pain state of the patient using the slope of the PSD of the sensed peripheral LFP signal.

In Example 7, the subject matter of one or any combination of Examples 1-6 optionally includes sensing a peripheral LFP signal from vagus nerve tissue of the patient; determining the slope of the PSD of the sensed peripheral LFP signal sensed from the vagus nerve tissue, and determining vagal tone of the patient using the slope of the PSD of the sensed peripheral LFP signal.

In Example 8, the subject matter of one or any combination of Examples 1-7 optionally includes determining the slope of the PSD of a gamma electroencephalography frequency band of the sensed LFP signal.

Example 9 includes subject matter (such as a neurostimulation device) or can optionally be combined with one or any combination of Examples 1-8 to include such subject matter, comprising a sensing circuit configured to sense sensing a local field potential (LFP) signal of a patient when connected to implantable electrodes and signal processing circuitry operatively coupled to the sensing circuit. The signal processing circuitry is configured to compute a power spectral density (PSD) of the sensed LFP signal, compute a slope of the PSD of the sensed LFP signal, and determine a physiological state of the patient using the computed slope of the PSD of the sensed LFP signal.

In Example 10, the subject matter of Example 9 optionally includes signal processing circuitry configured to detect a movement state indicative of tremor movement of the patient using the slope of the PSD of the sensed LFP signal, a stimulation circuit configured to deliver electrical neurostimulation therapy to the patient when connected to the implantable electrodes, and a control circuit operatively coupled to the stimulation circuit and the signal processing circuitry. The control circuit is configured to change a neurostimulation therapy parameter based on the detected movement state of the patient.

In Example 11, the subject matter of one or both of Examples 9 and 10 optionally includes signal processing circuitry configured to determine a medication state of the patient using the slope of the PSD of the sensed LFP signal, and generate a prompt related to medication of the patient based on the determined medication state of the patient.

In Example 12, the subject matter of Example 9 optionally includes signal processing circuitry configured to detect a sleep state of the patient using the slope of the PSD of the sensed LFP signal, wherein the sleep state is indicative of depth of the sleep of the patient; a stimulation circuit configured to deliver electrical neurostimulation therapy to the patient when connected to the implantable electrodes; and a control circuit operatively coupled to the stimulation circuit and the signal processing circuitry. The control circuit is configured to control delivery of the neurostimulation therapy to the patient; and change a neurostimulation therapy parameter based on the detected sleep state of the patient.

In Example 13, the subject matter of Example 9 optionally includes signal processing circuitry configured to detect a change in impairment state of the patient using the slope of the PSD of the sensed LFP signal; a stimulation circuit configured to deliver electrical neurostimulation therapy to the patient when connected to the implantable electrodes; and a control circuit operatively coupled to the stimulation circuit and the signal processing circuitry. The control circuit is configured to control delivery of the neurostimulation therapy to the patient; and change a neurostimulation therapy parameter based on the detected change in impairment state of the patient.

In Example 14, the subject matter of one or any combination of Examples 9-13 optionally includes a sensing circuit configured to sense a spinal LFP signal from spinal nerve tissue of the patient, and signal processing circuitry is configured to determine the slope of the PSD of the sensed spinal LFP signal.

In Example 15, the subject matter of one or any combination of Examples 9-14 optionally includes a sensing circuit configured to sense a peripheral LFP signal from peripheral nerve tissue peripheral to a spine of the patient, and signal processing circuitry configured to determine the slope of the PSD of the sensed peripheral LFP signal.

In Example 16, the subject matter of one or any combination of Examples 9-15 optionally includes a sensing circuit configured to sense a peripheral LFP signal from peripheral nerve tissue peripheral to a brain of the patient; and signal processing circuitry configured to determine the slope of the PSD of the sensed peripheral LFP signal.

In Example 17, the subject matter of one or any combination of Examples 9-16 optionally includes signal processing circuitry configured to calculate the slope of the PSD of a gamma electroencephalography frequency band of the sensed LFP signal; and determine the physiological state of the patient using the calculated slope of the PSD of the gamma electroencephalography frequency band of the sensed LFP signal.

Example 18 includes subject matter (or can optionally be combined with one or any combination of Examples 1-17 to include such subject matter) such as a computer readable storage medium including instructions that when operated on by a medical device cause the medical device to perform acts comprising sensing a local field potential (LFP) signal of a patient using the medical device; calculating a power spectral density (PSD) of the sensed LFP signal and a slope of the PSD of the sensed LFP signal; and determining a physiological state of the patient using the slope of the PSD of the sensed LFP signal.

In Example 19, the subject matter of Example 18 optionally includes a computer readable storage medium including instructions that cause the medical device to perform acts including delivering electrical neurostimulation therapy to the patient; and changing at least one neurostimulation therapy parameter according to the physiological state of the patient determined using the slope of the PSD of the sensed LFP signal.

In Example 20, the subject matter of Example 18 optionally includes a computer readable storage medium including instructions that cause the medical device to perform acts including determining at least one of a sleep state, movement state, medication state, or disease impairment state of the patient using the slope of the PSD of the sensed LFP signal.

These non-limiting examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of a neurostimulation system.

FIG. 2 is an illustration of portions of another example of a neurostimulation system.

FIG. 3 is an illustration of an example of an implantable pulse generator (IPG) and an implantable lead system.

FIG. 4 is an illustration of another example of an IPG and an implantable lead system.

FIG. 5 is a schematic side view of an example of an electrical stimulation lead.

FIGS. 6A-6H are illustrations of an example of electrodes of a stimulation lead.

FIG. 7 is a block diagram of portions of an example of a medical device.

FIG. 8 is a flow diagram of an example of a method to operate a medical device to monitor electrical neural activity.

FIG. 9 is an example of graphs of power spectral distribution for local filed potentials sensed in the subthalamic nucleus of a subject that is under anesthesia.

FIG. 10 is an example of graphs of power spectral distribution for local filed potentials sensed in the globus pallidus internus of a subject that is under anesthesia.

FIG. 11 is an illustration of operation of an implantable pulse generator and a personal patient device.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses devices, systems and methods for programming and delivering electrical neurostimulation to a patient or subject. Advancements in neuroscience and neurostimulation research have led to a demand for delivering complex patterns of neurostimulation energy for various types of therapies. The present system may be implemented using a combination of hardware and software designed to apply any neurostimulation (neuromodulation) therapy, including but not being limited to DBS, SCS, PNS, FES, Occipital Nerve Stimulation (ONS), Sacral Nerve Stimulation (SNS), and Vagus Nerve Stimulation (VNS) therapies.

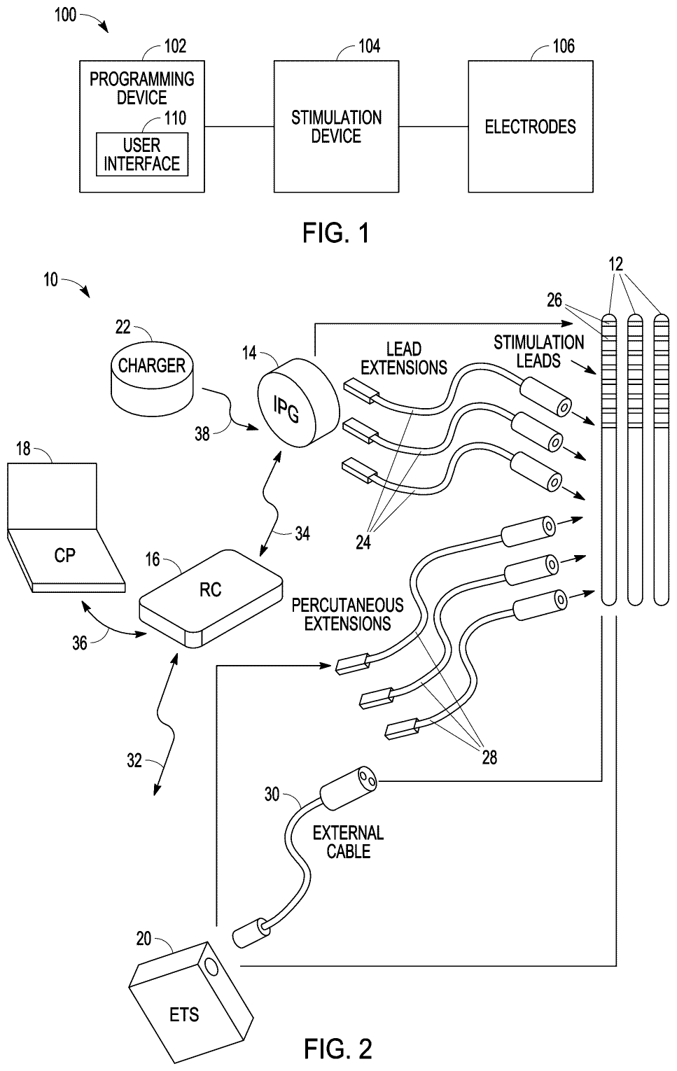

FIG. 1 illustrates an example of portions of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using multiple stimulation parameters, such as stimulation parameters specifying a pattern of the electrical pulses and a selection of electrodes through which each of the electrical pulses is delivered. In various embodiments, at least some of the stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device 104 via a wired or wireless link.

In this document, a “user” includes a physician or other clinician or caregiver who treats the patient using system 100; a “patient” includes a person who receives or is intended to receive neurostimulation delivered using system 100. In various embodiments, the patient can be allowed to adjust their treatment using system 100 to certain extent, such as by adjusting certain therapy parameters and entering feedback and clinical effect information.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various stimulation waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for DBS applications. The DBS configurations include various features that may simplify the task of the user in programming the stimulation device 104 for delivering DBS to the patient, such as the features discussed in this document.

FIG. 2 is an illustration of portions of another example of a neurostimulation system 10 that includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG 14 can optionally be physically connected via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The implantable pulse generator can have multiple stimulation channels (e.g., 8 or 16) which may be independently programmable to control the magnitude of the current stimulus from each channel. The IPG 14 can have one, two, three, four, or more connector ports, for receiving the terminals of the leads 12.

The ETS 20 may also be physically connected, optionally via the percutaneous lead extensions 28 and external cable 30, to the stimulation leads 12. The ETS 20, which may have similar pulse generation circuitry as the IPG 14, can also deliver electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a wireless communications link 32. Once the IPG 14 and neurostimulation leads 12 are implanted, the RC 16 may be used to telemetrically communicate with or control the IPG 14 via communications link 34. The communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

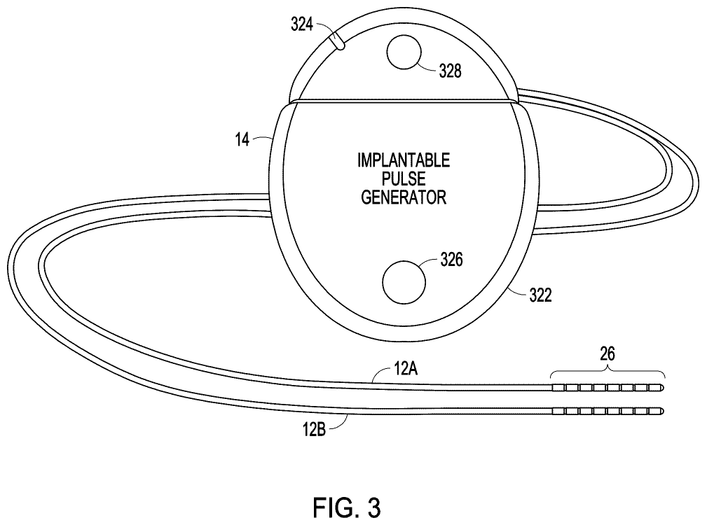

FIG. 3 is an illustration of an example of an IPG 14 (e.g., IPG 14 in FIG. 2) and an implantable lead system that includes stimulation leads (e.g., stimulation leads 12 in FIG. 2). The IPG 14 can be used as stimulation device 104 in FIG. 1. As illustrated in FIG. 3, IPG 14 that can be coupled to implantable leads 12A and 12B at a proximal end of each lead. The distal end of each lead includes electrical contacts or electrodes 26 for contacting a tissue site targeted for electrical neurostimulation. As illustrated in FIG. 3, leads 12A and 12B each include 8 electrodes 26 at the distal end. The number and arrangement of leads 12A and 12B and electrodes 26 as shown in FIGS. 2 and 3 are only examples, and other numbers and arrangements are possible. In various examples, the lead electrodes 26 are ring electrodes. In various examples the lead electrodes 26 include one or more segmented electrodes.

The IPG 14 can include a hermetically sealed IPG case 322 to house the electronic circuitry of IPG 14. IPG 14 can include an electrode 326 formed on IPG case 322. IPG 14 can include an IPG header 324 for coupling the proximal ends of leads 12A and 12B. IPG header 324 may optionally also include an electrode 328. One or both of electrodes 326 and 328 may be used as a reference electrode.

The implantable leads and electrodes may be configured by shape and size to provide electrical neurostimulation energy to a neuronal target included in the subject's brain. Neurostimulation energy can be delivered in a monopolar (also referred to as unipolar) mode using electrode 326 or electrode 328 and one or more electrodes selected from electrodes 26. Neurostimulation energy can be delivered in a bipolar mode using a pair of electrodes of the same lead (lead 12A or lead 12B). Neurostimulation energy can be delivered in an extended bipolar mode using one or more electrodes of a lead (e.g., one or more electrodes of lead 12A) and one or more electrodes of a different lead (e.g., one or more electrodes of lead 12B).

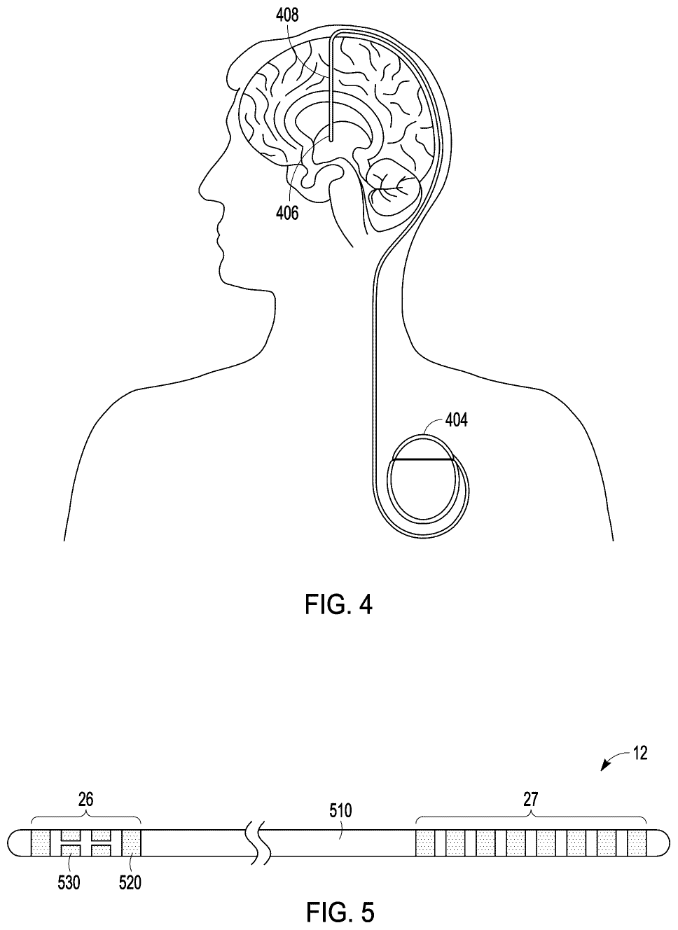

FIG. 4 illustrates another example of an IPG 404 and an implantable lead system 408 arranged to provide neurostimulation to a patient. An example of IPG 404 includes IPG 14 of FIGS. 2 and 3. An example of lead system 408 includes one or more of leads 12A and 12B in FIG. 3. The distal end 406 of the lead includes multiple electrodes (e.g., electrodes 26 in FIG. 3). In the illustrated embodiment, implantable lead system 408 is arranged to provide Deep Brain Stimulation (DBS) to a patient, with the stimulation target being neuronal tissue in a subdivision of the thalamus of the patient's brain. Other examples of DBS targets include neuronal tissue of the globus pallidus (GPi), the subthalamic nucleus (STN), the pedunculopontine nucleus (PPN), substantia nigra pars reticulate (SNr), cortex, globus pallidus externus (GPe), medial forebrain bundle (MFB), periaquaductal gray (PAG), periventricular gray (PVG), habenula, subgenual cingulate, ventral intermediate nucleus (VIM), anterior nucleus (AN), other nuclei of the thalamus, zona incerta, ventral capsule, ventral striatum, nucleus accumbens, and any white matter tracts connecting these and other structures.

After implantation, a clinician will program the neurostimulation device 400 using a CP 18, remote control, or other programming device. The programmed neurostimulation device 400 can be used to treat a neurological condition of the patient, such as Parkinson's Disease, Tremor, Epilepsia, Alzheimer's Disease, other Dementias, Stroke, Multiple Sclerosis, Amyotrophic Lateral Sclerosis (ALS), Autism, brain injury, brain tumor, migraine or other pain or headache condition, and any neurological syndromes that are congenic, degenerative, or acquired.

Returning to FIG. 3, the electronic circuitry of IPG 14 can include a stimulation control circuit that controls delivery of the neurostimulation energy. The stimulation control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off”′ modulation time), amplitudes of pulses in the pulse train, polarity of the pulses, etc.

FIG. 5 is a schematic side view of an embodiment of an electrical stimulation lead. FIG. 5 illustrates a stimulation lead 12 with electrodes 26 disposed at least partially about a circumference of the lead 12 along a distal end portion of the lead and terminals 27 disposed along a proximal end portion of the lead. The lead 12 can be implanted near or within the desired portion of the body to be stimulated (e.g., the brain, spinal cord, or other body organs or tissues). In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 12 can be inserted into the cranium and brain tissue with the assistance of a stylet (not shown). The lead 12 can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): insert the lead 12, advance the lead 12, retract the lead 12, or rotate the lead 12.

In some embodiments, measurement devices coupled to the muscles or other tissues stimulated by the target neurons, or a unit responsive to the patient or clinician, can be coupled to the implantable pulse generator or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

The lead 12 for deep brain stimulation can include stimulation electrodes, recording electrodes, or both. In at least some embodiments, the lead 12 is rotatable so that the stimulation electrodes can be aligned with the target neurons after the neurons have been located using the recording electrodes. Stimulation electrodes may be disposed on the circumference of the lead 12 to stimulate the target neurons. Stimulation electrodes may be ring-shaped so that current projects from each electrode equally in every direction from the position of the electrode along a length of the lead 12. In the embodiment of FIG. 5, two of the electrodes 520 are ring electrodes 520. Ring electrodes typically do not enable stimulus current to be directed from only a limited angular range around of the lead. Segmented electrodes 530, however, can be used to direct stimulus current to a selected angular range around the lead. When segmented electrodes 530 are used in conjunction with an IPG 14 that delivers constant current stimulus, current steering can be achieved to more precisely deliver the stimulus to a position around an axis of the lead (e.g., radial positioning around the axis of the lead). To achieve current steering, segmented electrodes can be utilized in addition to, or as an alternative to, ring electrodes.

The lead 12 includes a lead body 510, terminals 27, and one or more ring electrodes 520 and one or more sets of segmented electrodes 530 (or any other combination of electrodes). The lead body 510 can be formed of a biocompatible, non-conducting material such as, for example, a polymeric material. Suitable polymeric materials include, but are not limited to, silicone, polyurethane, polyurea, polyurethaneurea, polyethylene, or the like. Once implanted in the body, the lead 12 may be in contact with body tissue for extended periods of time. In at least some embodiments, the lead 12 has a cross-sectional diameter of no more than 1.5 millimeters (1.5 mm) and may be in the range of 0.5 to 1.5 mm. In at least some embodiments, the lead 12 has a length of at least 10 centimeters (10 cm) and the length of the lead 12 may be in the range of 10 to 70 cm.

The electrodes 26 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use. Each of the electrodes can either be used or unused (OFF). When the electrode is used, the electrode can be used as an anode or cathode and carry anodic or cathodic current. In some instances, an electrode might be an anode for a period of time and a cathode for a period of time.

Deep brain stimulation leads and other leads may include one or more sets of segmented electrodes. Segmented electrodes may provide for superior current steering than ring electrodes because target structures in deep brain stimulation or other stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue.

Any number of segmented electrodes 530 may be disposed on the lead body 510 including, for example, anywhere from one to sixteen or more segmented electrodes 530. It will be understood that any number of segmented electrodes 530 may be disposed along the length of the lead body 510. A segmented electrode 530 typically extends only 75%, 67%, 60%, 50%, 40%, 33%, 25%, 20%, 17%, 15%, or less around the circumference of the lead.

The segmented electrodes 530 may be grouped into sets of segmented electrodes, where each set is disposed around a circumference of the lead 12 at a particular longitudinal portion of the lead 12. The lead 12 may have any number segmented electrodes 530 in a given set of segmented electrodes. The lead 12 may have one, two, three, four, five, six, seven, eight, or more segmented electrodes 530 in a given set. In at least some embodiments, each set of segmented electrodes 530 of the lead 12 contains the same number of segmented electrodes 530. The segmented electrodes 530 disposed on the lead 12 may include a different number of electrodes than at least one other set of segmented electrodes 530 disposed on the lead 12. The segmented electrodes 530 may vary in size and shape. In some embodiments, the segmented electrodes 530 are all of the same size, shape, diameter, width or area or any combination thereof. In some embodiments, the segmented electrodes 530 of each circumferential set (or even all segmented electrodes disposed on the lead 12) may be identical in size and shape.

Each set of segmented electrodes 530 may be disposed around the circumference of the lead body 510 to form a substantially cylindrical shape around the lead body 510. The spacing between individual electrodes of a given set of the segmented electrodes may be the same, or different from, the spacing between individual electrodes of another set of segmented electrodes on the lead 12. In at least some embodiments, equal spaces, gaps or cutouts are disposed between each segmented electrode 530 around the circumference of the lead body 510. In other embodiments, the spaces, gaps or cutouts between the segmented electrodes 530 may differ in size, or cutouts between segmented electrodes 530 may be uniform for a particular set of the segmented electrodes 530 or for all sets of the segmented electrodes 530. The sets of segmented electrodes 530 may be positioned in irregular or regular intervals along a length the lead body 510.

Conductor wires (not shown) that attach to the ring electrodes 520 or segmented electrodes 530 extend along the lead body 510. These conductor wires may extend through the material of the lead 12 or along one or more lumens defined by the lead 12, or both. The conductor wires couple the electrodes 520, 530 to the terminals 27.

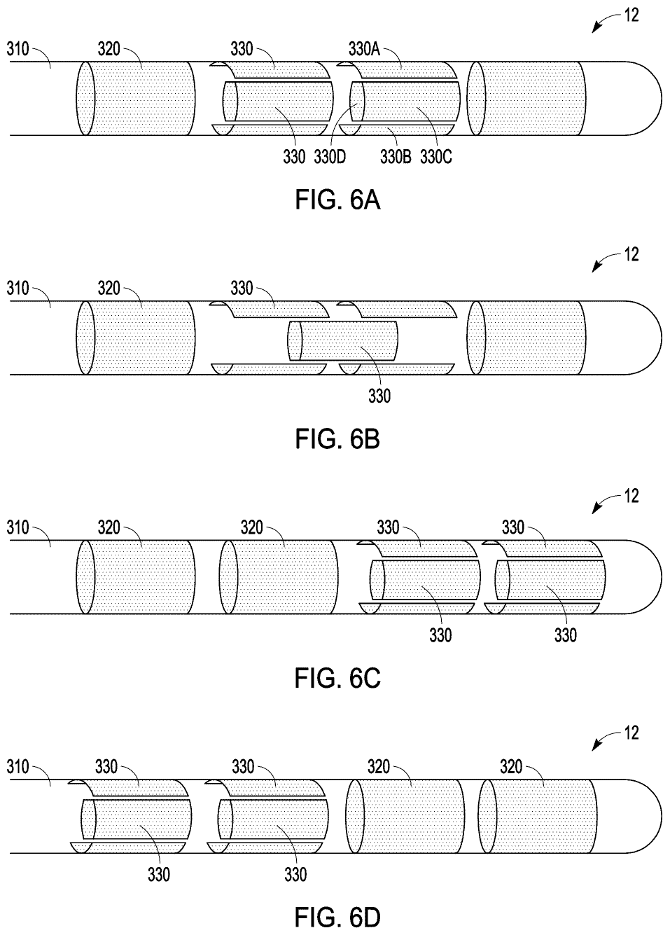

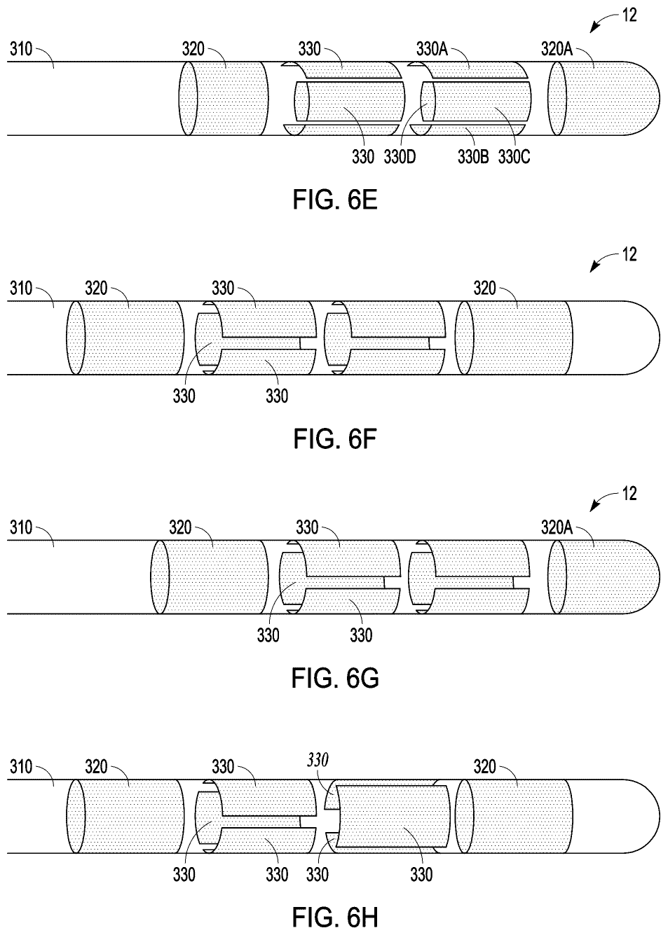

FIGS. 6A-6H are illustrations of different embodiments of leads 12 with segmented electrodes 330, optional ring electrodes 320 or tip electrodes 320a, and a lead body 310. The sets of segmented electrodes 330 each include either two (FIG. 6B), three (FIGS. 6E-6H), or four (FIGS. 6A, 6C, and 6D) or any other number of segmented electrodes including, for example, three, five, six, or more. The sets of segmented electrodes 330 can be aligned with each other (FIGS. 6A-6G) or staggered (FIG. 6H).

When the lead 12 includes both ring electrodes 320 and segmented electrodes 330, the ring electrodes 320 and the segmented electrodes 330 may be arranged in any suitable configuration. For example, when the lead 12 includes two ring electrodes 320 and two sets of segmented electrodes 330, the ring electrodes 120 can flank the two sets of segmented electrodes 330 (see e.g., FIGS. 5, 6A, and 6E-6H, ring electrodes 320 and segmented electrode 330). Alternately, the two sets of ring electrodes 320 can be disposed proximal to the two sets of segmented electrodes 330 (see e.g., FIG. 6C, ring electrodes 320 and segmented electrode 330), or the two sets of ring electrodes 320 can be disposed distal to the two sets of segmented electrodes 330 (see e.g., FIG. 6D, ring electrodes 320 and segmented electrode 330). One of the ring electrodes can be a tip electrode (see e.g., tip electrode 320a of FIGS. 6E and 6G). It will be understood that other configurations are possible as well (e.g., alternating ring and segmented electrodes, or the like).

By varying the location of the segmented electrodes 330, different coverage of the target neurons may be selected. For example, the electrode arrangement of FIG. 6C may be useful if the physician anticipates that the neural target will be closer to a distal tip of the lead body 310, while the electrode arrangement of FIG. 6D may be useful if the physician anticipates that the neural target will be closer to a proximal end of the lead body 310.

Any combination of ring electrodes 320 and segmented electrodes 330 may be disposed on the lead 12. For example, the lead 12 may include a first ring electrode 320, two sets of segmented electrodes; each set formed of four segmented electrodes 330, and a final ring electrode 320 at the end of the lead. This configuration may simply be referred to as a 1-4-4-1 configuration (FIGS. 6A and 6E, ring electrodes 320 and segmented electrode 330). It may be useful to refer to the electrodes with this shorthand notation. Thus, the embodiment of FIG. 6C may be referred to as a 1-1-4-4 configuration, while the embodiment of FIG. 6D may be referred to as a 4-4-1-1 configuration. The embodiments of FIGS. 6F, 6G, and 6H can be referred to as a 1-3-3-1 configuration. Other electrode configurations include, for example, a 2-2-2-2 configuration, where four sets of segmented electrodes are disposed on the lead, and a 4-4 configuration, where two sets of segmented electrodes, each having four segmented electrodes 330 are disposed on the lead. The 1-3-3-1 electrode configuration of FIGS. 6F, 6G, and 6H has two sets of segmented electrodes, each set containing three electrodes disposed around the circumference of the lead, flanked by two ring electrodes (FIGS. 6F and 6H) or a ring electrode and a tip electrode (FIG. 6G). In some embodiments, the lead includes 16 electrodes. Possible configurations for a 16-electrode lead include but are not limited to 4-4-4-4; 8-8; 3-3-3-3-3-1 (and all rearrangements of this configuration); and 2-2-2-2-2-2-2-2.

Any other suitable arrangements of segmented and/or ring electrodes can be used. As an example, arrangements in which segmented electrodes are arranged helically with respect to each other. One embodiment includes a double helix. One or more electrical stimulation leads can be implanted in the body of a patient (for example, in the brain or spinal cord of the patient) and used to stimulate surrounding tissue. The lead(s) are coupled to the implantable pulse generator (such as IPG 14 in FIG. 2).

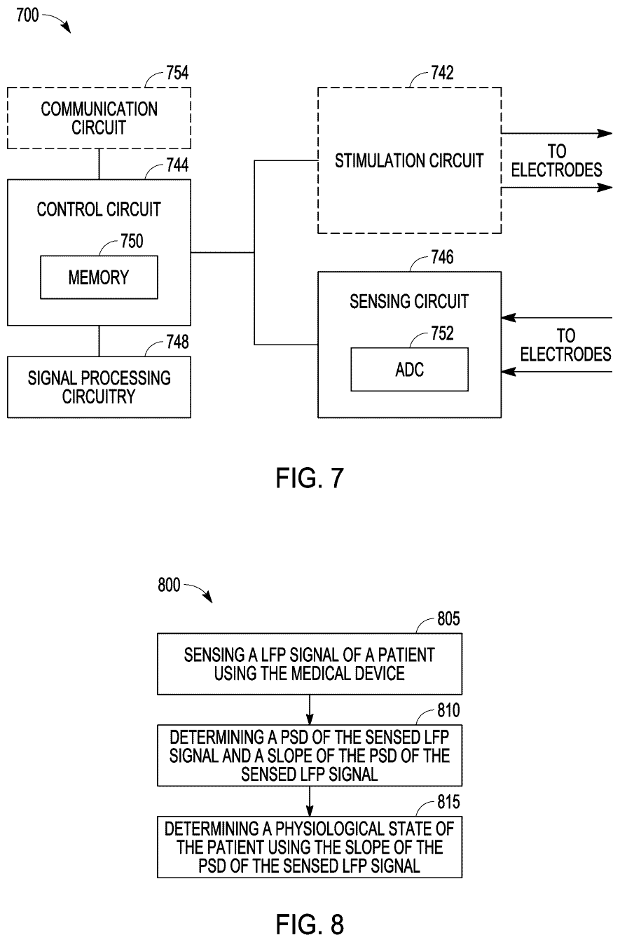

FIG. 7 is a block diagram of portions of an embodiment of a medical device 700. The medical device 700 may be a monitoring device to monitor neural activity signals of a patient or subject, or the medical device 700 may be a neurostimulation device (e.g., the IPG 14 of FIG. 2) for providing neurostimulation to a patient. The medical device 700 includes a control circuit 744 and a sensing circuit 746. The sensing circuit 746 can include one or more sense amplifiers to sense internal neural signals of the patient. The sense amplifiers may be switchable among recording electrodes. The control circuit 744 can include processing circuitry such as a microprocessor, a digital signal processor, application specific integrated circuit (ASIC), or other type of processor, interpreting or executing instructions in software modules or firmware modules. The instructions can be stored in memory 750 that can be integral to the control circuit 744 or separate from the control circuit 744. The control circuit 744 can include other circuits or sub-circuits to perform the functions described. These circuits may include software, hardware, firmware, or any combination thereof. Multiple functions can be performed in one or more of the circuits or sub-circuits as desired.

The medical device 700 can also include a communicate circuit 754 to communicate wirelessly with a separate device (e.g., CP 18 in FIG. 2). If the medical device 700 is a neurostimulation device, the medical device 700 includes a stimulation circuit 742. The stimulation circuit 742 can be operatively coupled to stimulation electrodes such as any of the electrodes and leads described herein and the stimulation circuit 742 provides or delivers electrical neurostimulation energy to the electrodes. The control circuit 744 can control delivery of electrical neurostimulation using the stimulation circuit 742. The electrical neurostimulation may be delivered according to neurostimulation therapy parameters configured in the control circuit 744 (e.g., by programming).

The medical device 700 includes signal processing circuitry 748 that can be separate from the control circuit 744 or integral to the control circuit 744. The signal processing circuitry 748 can include one or more processes running on one or processors (e.g., one or more microprocessors that may include the control circuit 744 or may be separate from the control circuit 744) to perform signal analysis or other signal processing on the neural signals sensed using the sensing circuit 746. The medical device 700 can include an analog-to-digital converter (ADC) circuit 752 to digitize the sensed neural signals for signal processing.

The medical device 700 can sense electrical neural signals when coupled to the electrodes. The medical device 700 can be used to sense the response of the patient to neurostimulation. The sensed response can be used to adjust the neurostimulation by either an automatic adjustment of the parameters of the neurostimulation therapy or by recommending parameter settings to a user.

FIG. 8 is a flow diagram of an example of a method 800 of operating a medical device (e.g., the medical device 700 in FIG. 7) to monitor electrical neural activity of a patient or subject. The medical device computes and utilizes the low frequency power (e.g., 1/f power) of sensed neural signals to detect physiological conditions of the patient or physiological events that are relevant to the neuromodulation therapy. If the medical device provides neurostimulation, the 1/f power distribution can be utilized as part of an adaptive neuromodulation therapy provided by the medical device. The 1/f power distribution can be characterized by its slope, and the slope of the 1/f power can be a robust measure that is therapeutically relevant to the physiological condition of a patient.

At block 805, a local field potential (LFP) signal of a patient is sensed using a sensing circuit of the medical device (e.g., sensing circuit 746 in FIG. 7). The medical device may be connected to DBS electrodes and the LFP signal can be sensed from brain tissue. For example, the LFP signal may be sensed using electrodes on or near the subthalamic nucleus (STN), the globus pallidus internus (GPi), or the motor cortex of the patient. In certain examples, the LFP signal is an electroencephalogram (EEG) signal. The sensing circuitry may sense an LFP signal having a frequency in one or more of the alpha frequency band, the beta frequency band, the gamma frequency band, or the theta frequency band of the electroencephalography frequency bands.

In some examples, the medical device is connected to SCS electrodes and the LFP signal is sensed from spinal nerve tissue of the patient. In some examples, the medical device is connected to PNS electrodes and the LFP signal is sensed from nerve tissue (e.g., the vagus nerve of the patient). In some examples, the medical device senses an LFP signal peripheral to the brain of the patient.

At block 810, the signal processing circuitry of the medical device (e.g., signal processing circuitry 748 in FIG. 7) computes the power spectral density (PSD) of one or more sensed LFP signals. In some examples, the signal processing circuitry computes a Fast Fourier Transform (FFT) of the LFP signals or a short time Fourier Transform (STFT) to obtain a 1/f power distribution of the LFP signals. In certain examples, the signal processing circuitry performs an auto correlation function (ACF) analysis of the LFP signals, or a wavelet spectral analysis of the LFP signal. The signal processing circuitry computes the slope of the PSD determined from the LFP signals.

The medical device may use other methods to compute the PSD. In some examples, the medical device calculates an estimate of the PSD using Welch's method and computes the slope of the estimated PSD. In some examples, the medical device implements a neural network or other machine learning algorithm. The neural network may be trained to output the PSD or the slope of the PSD from one or more LFP signals. The neural network may determine the PSD for a band or range of frequencies.

At block 815, the medical device determines a physiological state of the patient using the computed slope of the PSD of the sensed LFP signal. The quantified PSD slope is linked to the degree of excitation/inhibition (E/I) balance present in the neural activity reflected in the LFP signal. An example of a physiological state detectable by the medical device from the slope of the PSD of the sensed LFP signal is a sleep state of the patient from normal sleep or from anesthesia. The medical device can determine if the patient is in a deep sleep state or a light sleep state from the slope of the PSD of the sensed LFP signal.

In another example, the medical device determines a pain state of the patient. The LFP signal may sensed from spinal nerve tissue of the patient and the medical device determines a change in the paint state of the patient using the computed slope of the PSD of the sensed spinal LFP signal. In a further example, the medical device senses an LFP signal from nerve tissue peripheral to the spine of the patient and the medical device determines a physiological state of the patient using the slope of the PSD of the sensed peripheral LFP signal. For instance, the LFP signal may be sensed from a vagus nerve of the patient, and determines vagal tone of the patient using the computed slope of the PSD of the LFP signal sensed from the vagus nerve.

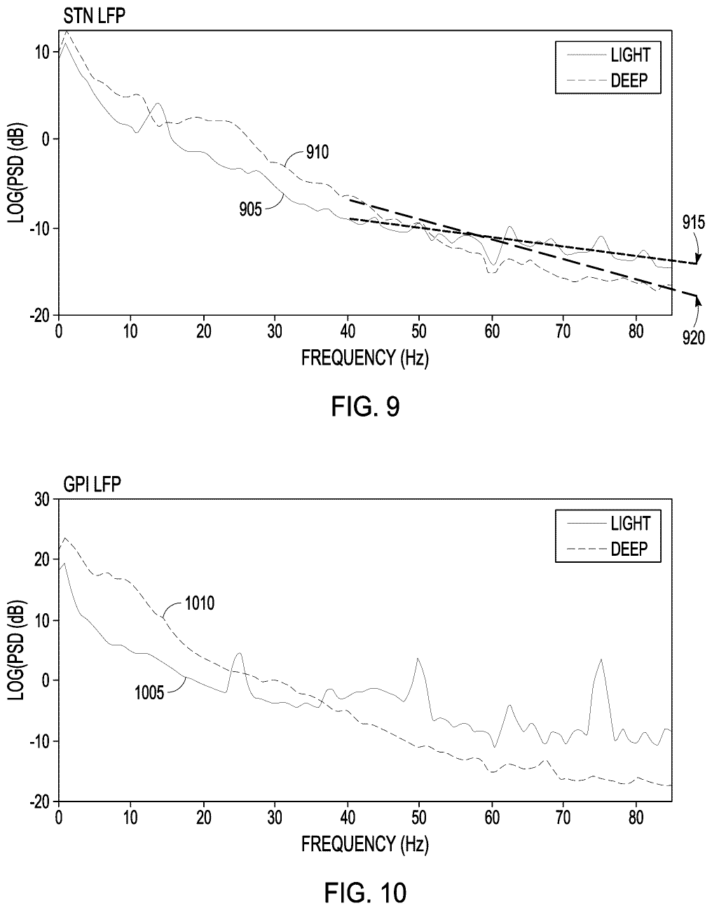

FIG. 9 is an example of two graphs of the PSD distribution for LFPs sensed in the STN of a subject that is under anesthesia. The graph 905 represents PSD in decibels (dB) versus frequency for the subject in a lighter anesthetic state and graph 910 represents PSD in dB versus frequency for the subject under a deeper anesthetic state. The graphs show that above about 45 Hertz (45 Hz) the PSD slope 920 for the deeper anesthetic state is steeper than the PSD slope 915 for the lighter anesthetic state. Thus, the steeper PSD slope reflects greater inhibition of neural activity. The signal processing circuitry may calculate the PSD slope as a ratio of the change in power over the change in frequency. In some examples, the signal processing circuitry estimated the PSD slope as a ratio of average power over a first frequency range (e.g., P_{AVE} over 70-90 Hz) over the average power over a second frequency range (e.g., P_{AVE} over 50-70 Hz).

FIG. 10 is an example of two graphs of the PSD distribution for LFPs sensed in the GPi of a subject that is under anesthesia. The graphs show that above about 35 Hz the PSD slope of the graph 1010 for the deeper anesthetic state is steeper than the PSD slope 1005 for the lighter anesthetic state, and again the steeper slope reflects greater inhibition of neural activity. The LFP signals may be sensed from other areas of interest (e.g., the motor cortex) and the slope of the PSD determined by the medical device can be used to distinguish between physiological states having different degrees of excitation or inhibition of neural activity, such as distinguish between a deeper sleep state and a lighter sleep state. If the medical device provides neurostimulation therapy, the control circuit of the medical device (e.g., control circuit 744 in FIG. 7) may perform a decision (e.g., to change a therapy parameter) to optimize the neurostimulation therapy.

Because medical device can calculate the PSD of LFP signals and quantify the PSD slope, the medical device can monitor PSD slope for a change in a disease state or impairment state of the patient. For example, the medical device may recurrently monitor the PSD slope and detect when the PSD slope has changed by more than a predetermined amount. When the change in PSD slope is detected, the medical device may send a prompt to another device alerting a user of the change in the patient's impairment. If the medical device provides neurostimulation therapy, the control circuit of the medical device may change a neurostimulation therapy parameter in response to the detected change in PSD slope.

The medical device can distinguish between other physiological states using the slope of the PSD distribution. For example, the patient may be being treated with medication. A steeper PSD slope detected by the medical device can indicate a higher level of inhibition of neural activity, indicating that the patient has taken the medication and the medication is working. A flatter slope detected by the medical device can indicate a lower level of inhibition of neural activity, indicating that the patient did not take the medication or that the patient should take another dose of the medication. The signal processing circuitry of the medical device may generate a prompt related to medication of the patient based on the detected medication state of the patient.

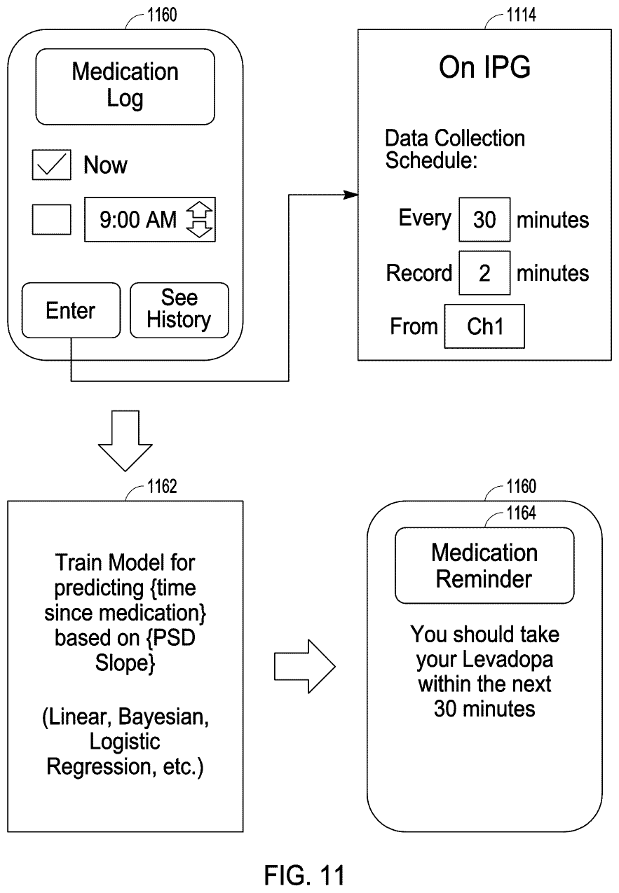

FIG. 11 is an illustration of operation of an IPG 1114 and a personal device of the patient, such as a smartphone 1160 for example. The smartphone 1160 includes a companion application (or app) for the IPG 1114. The app may be used to train a classifier or regression algorithm that can detect a medication state and provide information about the medication state of the patient.

The IPG 1114 collects PSD slope data according to a schedule (e.g., 2 minutes of LFP signals sensed every 30 minutes) and calculates PSD slope data from the sensed signals. The user of the smartphone (e.g., the patient or caregiver) logs medication events (e.g., when the patient took the medication, when the patient omitted taking the medication, etc.). The IPG 1114 pairs with the smartphone 1152 to transfer PSD slope data to the smartphone 1160. The app correlates the PSD slope data of the IPG 1114 with the logged medication events.

The PSD slope data and medication events can be compiled over many cycles of absorbing the medication and the medication wearing off. These cycles of data can be input as training data to a train a machine learning model 1162 (e.g., a linear regression model, a Bayesian model, a logistic regression model, etc.). The machine learning model 1162 may be deployed on the smartphone 1160. The machine learning model 1162 can provide informed medication reminders 1164 on the smartphone 1160 to the patient based on subsequent PSD slope data.

In another example, the machine learning model 1162 is deployed in the IPG 1114. The logged medication events are transferred to the IPG 1114, and the cycles of medication and PSD slope data are input to the machine learning model 1162. The trained model may be deployed in the IPG 1114 (e.g., in IPG firmware) and the IPG 1114 may send medication reminders to the smartphone 1160 based on subsequent PSD slope data collected by the IPG.

In some examples, sensors can be used to provide additional context to the PSD slope information obtained from the LFP signals. For example, the PSD slope information may be used to detect a movement state of the patient, such as if the patient is experiencing tremors for example. A flatter PSD slope may indicate more movement than a steeper PSD slope indicating inhibition in neural activity causing the movement. A motion sensor such as an accelerometer (wearable or integrated into the medical device) can be used to confirm the type of movement associated with patient's impairment or disease and confirm the movement state detected form PSD slope information. If the medical device provides neurostimulation therapy, the control circuit of the medical device may change a therapy parameter based on the detected movement state of the patient.

Other wearable or implantable sensors can provide information regarding change in the impairment state of the patient, such as sensors that can provide information regarding changes in the patient's speech due to increase in impairment from the disease for example. The additional information from the additional sensor or sensors can lead to a more accurate device-based assessment of the patient's condition.

The systems and methods described herein include techniques for improving performance of adaptive neuromodulation approaches. The 1/f power distribution can provide robust feedback for a closed-loop neuromodulation therapy.

The above detailed description is intended to be illustrative, and not restrictive. The scope of the disclosure should, therefore, be determined with references to the appended claims, along with the full scope of equivalents to which such claims are entitled.

What is claimed is:

1. A computer-implemented method of operating a medical device, the method comprising:
sensing a local field potential (LFP) signal of a patient using the medical device;
determining, by the medical device, a power spectral density (PSD) of the sensed LFP signal and a slope of the PSD of the sensed LFP signal; and
determining a physiological state of the patient using the slope of the PSD of the sensed LFP signal.

2. The method of claim 1, including:
delivering electrical neurostimulation therapy to the patient using the medical device;
detecting a movement state of the patient using the slope of the PSD of the sensed LFP signal, wherein the movement state is indicative of tremor movement of the patient; and
changing a neurostimulation therapy parameter based on the detected movement state of the patient.

3. The method of claim 1, including:
determining a medication state of the patient using the slope of the PSD of the sensed LFP signal; and
generating a prompt related to medication of the patient based on the determined medication state of the patient.

4. The method of claim 1, including:
delivering electrical neurostimulation therapy to the patient using the medical device;
detecting a sleep state of the patient using the slope of the PSD of the sensed LFP signal, wherein the sleep state is indicative of depth of the sleep of the patient; and
changing a neurostimulation therapy parameter based on the detected sleep state of the patient.

5. The method of claim 1, including:
delivering electrical neurostimulation therapy to the patient using the medical device;
detecting a change in impairment state of the patient using the slope of the PSD of the sensed LFP signal; and
changing a neurostimulation therapy parameter based on the detected change in impairment state of the patient.

6. The method of claim 1,
wherein the sensing the LFP signal includes sensing a spinal LFP signal from spinal nerve tissue of the patient;
wherein the determining the slope of the PSD includes determining the slope of the PSD of the sensed spinal LFP signal; and
determining a change in a pain state of the patient using the slope of the PSD of the sensed peripheral LFP signal.

7. The method of claim 1,
wherein the sensing the LFP signal includes sensing a peripheral LFP signal from vagus nerve tissue of the patient;
wherein the determining the slope of the PSD includes determining the slope of the PSD of the sensed peripheral LFP signal; and
determining vagal tone of the patient using the slope of the PSD of the sensed peripheral LFP signal.

8. The method of claim 1, wherein the determining the slope of the PSD includes determining the slope of the PSD of a gamma electroencephalography frequency band of the sensed LFP signal.

9. A neurostimulation device comprising:
a sensing circuit configured to sense sensing a local field potential (LFP) signal of a patient when connected to implantable electrodes; and
signal processing circuitry operatively coupled to the sensing circuit and configured to:
compute a power spectral density (PSD) of the sensed LFP signal;
compute a slope of the PSD of the sensed LFP signal; and
determine a physiological state of the patient using the computed slope of the PSD of the sensed LFP signal.

10. The neurostimulation device of claim 9, including:
a stimulation circuit configured to deliver electrical neurostimulation therapy to the patient when connected to the implantable electrodes; and
a control circuit operatively coupled to the stimulation circuit and the signal processing circuitry, and configured to control delivery of the neurostimulation therapy to the patient;
wherein the signal processing circuitry is configured to detect a movement state indicative of tremor movement of the patient using the slope of the PSD of the sensed LFP signal; and
wherein the control circuit is configured to change a neurostimulation therapy parameter based on the detected movement state of the patient.

11. The neurostimulation device of claim 9, wherein the signal processing circuitry is configured to:
determine a medication state of the patient using the slope of the PSD of the sensed LFP signal; and
generate a prompt related to medication of the patient based on the determined medication state of the patient.

12. The neurostimulation device of claim 9, including:
a stimulation circuit configured to deliver electrical neurostimulation therapy to the patient when connected to the implantable electrodes; and
a control circuit operatively coupled to the stimulation circuit and the signal processing circuitry, and configured to control delivery of the neurostimulation therapy to the patient;
wherein the signal processing circuitry is configured to detect a sleep state of the patient using the slope of the PSD of the sensed LFP signal, wherein the sleep state is indicative of depth of the sleep of the patient; and
wherein the control circuit is configured to change a neurostimulation therapy parameter based on the detected sleep state of the patient.

13. The neurostimulation device of claim 9, including:
a stimulation circuit configured to deliver electrical neurostimulation therapy to the patient when connected to the implantable electrodes; and
a control circuit operatively coupled to the stimulation circuit and the signal processing circuitry, and configured to control delivery of the neurostimulation therapy to the patient;
wherein the signal processing circuitry is configured to detect a change in impairment state of the patient using the slope of the PSD of the sensed LFP signal; and
wherein the control circuit is configured to change a neurostimulation therapy parameter based on the detected change in impairment state of the patient.

14. The neurostimulation device of claim 9,
wherein the sensing circuit is configured to sense a spinal LFP signal from spinal nerve tissue of the patient; and
wherein the signal processing circuitry is configured to determine the slope of the PSD of the sensed spinal LFP signal.

15. The neurostimulation device of claim 9,
wherein the sensing circuit is configured to sense a peripheral LFP signal from peripheral nerve tissue peripheral to a spine of the patient; and
wherein the signal processing circuitry is configured to determine the slope of the PSD of the sensed peripheral LFP signal.

16. The neurostimulation device of claim 9,
wherein the sensing circuit is configured to sense a peripheral LFP signal from peripheral nerve tissue peripheral to a brain of the patient; and
wherein the signal processing circuitry is configured to determine the slope of the PSD of the sensed peripheral LFP signal.

17. The neurostimulation device of claim 9, wherein the signal processing circuitry is configured to:
calculate the slope of the PSD of a gamma electroencephalography frequency band of the sensed LFP signal; and
determine the physiological state of the patient using the calculated slope of the PSD of the gamma electroencephalography frequency band of the sensed LFP signal.

18. A non-transitory computer readable storage medium including instructions that when operated on by a medical device cause the medical device to perform acts comprising:
sensing a local field potential (LFP) signal of a patient using the medical device;
calculating a power spectral density (PSD) of the sensed LFP signal and a slope of the PSD of the sensed LFP signal; and
determining a physiological state of the patient using the slope of the PSD of the sensed LFP signal.

19. The non-transitory computer readable storage medium of claim 18, including instructions that cause the medical device to perform acts including:
delivering electrical neurostimulation therapy to the patient; and
changing at least one neurostimulation therapy parameter according to the physiological state of the patient determined using the slope of the PSD of the sensed LFP signal.

20. The non-transitory computer readable storage medium of claim 18, including instructions that cause the medical device to perform acts including determining at least one of a sleep state, movement state, medication state, or disease impairment state of the patient using the slope of the PSD of the sensed LFP signal.
